(19) United States
(12) Patent Application Publication
(10) Pub. No.: US 2010/0209348 A1
(43) Pub. Date: Aug. 19, 2010

(54) METHODS FOR DETERMINING LIPOSOME BIOEQUIVALENCE

(75) Inventor: Francis J. Martin, San Francisco, CA (US)

Correspondence Address: WILSON, SONSINI, GOODRICH & ROSATI, 650 PAGE MILL ROAD, PALO ALTO, CA, 94304-1050, US

(73) Assignee: Arondo Pharma, Inc.

(21) Appl. No.: 12/487,039

(22) Filed: Jun. 18, 2009

Related U.S. Application Data
(60) Provisional application No. 61/153,599, filed on Feb. 18, 2009.

(51) Int. Cl. A61B 10/00; G01N 33/15; A61K 49/00

(52) U.S. Cl. 424/9.2; 436/93

(57) ABSTRACT

This invention provides methods for determining liposome bioequivalence between a generic drug product and a reference brand-name product. Specific methods for determining bioequivalence between doxorubicin hydrochloric acid (HCl) liposome injection product (Doxil®) and a generic pegylated liposome doxorubicin product are disclosed herein.

### CROSS REFERENCE

This application claims the benefit of U.S. Provisional Application No. 61/153,599, entitled “Method for Demonstrating Bioequivalence between a Reference Doxorubicin HCl Liposome Injection Product and a Generic Product” filed Feb. 18, 2009, which is incorporated herein in its entirety by reference.

### BACKGROUND OF THE INVENTION

Doxorubicin HCl (doxorubicin) is a widely prescribed chemotherapeutic agent that has been in clinical use for more than 30 years; it is often given in combination with other drugs to treat breast cancer, among many other tumor types. Although it enjoys a wide spectrum of anti-tumor activity, doxorubicin also causes toxicities to most patients who receive it. Toxicities are a consequence of the drug's tendency to quickly distribute indiscriminately to all body tissues where it can cause damage, particularly by killing normal cells that are continuously dividing, such as those in the bone marrow (which are constantly making new white blood cells), surface layers of the intestines and oral cavity (which are sloughed by physical forces and thereby constantly need to be replenished) and hair follicles (cells dividing rapidly in follicles to produce hair). Among the most serious adverse effects of doxorubicin therapy are decreases in certain types of white blood cells (including neutrophils, which can lead to transient immune-suppression and serious infections), nausea and vomiting, alopecia (hair loss) and damage to the heart muscle.

Pegylated Liposomal Doxorubicin (PLD) is a long-circulating liposomal formulation of doxorubicin. This type of liposome is designed to carry doxorubicin, entrapped within its aqueous “core”, through the bloodstream and to distribute it to tissues in encapsulated form.

PLD liposomes are made to be very small (about 85 nm; ˜100-times smaller than a red blood cell) and to circulate within the bloodstream for several days to a week. Long circulation is provided by a layer of polyethylene glycol (PEG) chemically grafted to the liposome surface. The PEG acts as an inert polymer layer that serves to mask the liposome from immune system recognition and uptake and/or destruction by components and dynamics inherent in the blood compartment. Some normal tissues and some tumors appear to have gaps, discontinuities or channels in the layer of cells (the endothelium) that surround the blood vessels that perfuse them. Over a several day period following intravenous administration, PLD liposomes apparently move through these gaps or channels and extravasate (leave the blood stream and enter the tissue compartment). Other mechanisms may also contribute to tissue uptake, such as transcytosis and phagocytosis by macrophages.

It is believed that the liposomes become physically trapped in the spaces between tumor cells (the interstitium) following entry into tumors. Retention in the tumor is believed to be related to the discovery that tumors generally lack lymphatic vessels, which in normal tissues serve to clear entering foreign particles. Liposomes entrapped in tumor eventually release drug that is then free to enter and kill nearby tumor cells. In contrast to tumor tissue, liposomes that enter normal tissues appear to become entrained in the flow of interstitial fluid and cleared by lymphatic drainage, a process that may take several days. Drug release likely occurs during liposome passage through normal tissues, as evidenced by the emergence of toxicities associated with PLD therapy.

The FDA authorized designation of the only commercial version of PLD approved in the United States is “Doxorubicin HCl Liposome Injection”. The drug product was approved in 1995 and is now marketed as Doxil in the United States by Ortho Biotech/JNJ and as Caelyx® in the rest of the world (ROW) by Schering Plough (S-P). As of January, 2009, the product is indicated world-wide for Kaposi's sarcoma and ovarian carcinoma and in the US for multiple myeloma (in combination with bortezomib). In the EU, Caelyx is also approved for breast carcinoma in patients at risk of anthracycline cardiotoxicity. The product offers recognized clinical benefit and occupies a unique clinical niche (O'Brien 2008). Ortho/JNJ submitted a supplemental New Drug Application (“SNDA”) in September, 2008 seeking US approval for Doxil in combination with docetaxel to treat recurrent breast carcinoma in anthracycline-exposed patients. The product is protected in the U.S. under U.S. Pat. No. 5,316,771 assigned to an affiliate of Johnson and Johnson, which expires in 2011. Other exclusivity may apply for orphan drug indications.

The United States Food and Drug Administration (FDA) recognizes the public policy benefit of generic competition but has understandably embraced safeguards to protect the public from generics that do not meet quality standards and/or are not objectively bioequivalent to the brand-name product. Rather than requiring a generic manufacturer to repeat the costly and time-consuming NDA process, the Drug Price Competition and Patent Term Restoration Act of 1984, (also known as the Waxman-Hatch Act) permits the sponsor to file an Abbreviated New Drug Application (“ANDA”). The object of the ANDA process is to demonstrate that the generic drug product has the same active ingredient, route of administration, dosage form and strength, and proposed labeling as the brand-name drug. The ANDA also must contain sufficient information to demonstrate that the generic drug is “bioequivalent” to the relevant brand-name product. When acceptable information of this type is provided, the generic applicant is permitted to rely on the FDA's previous findings of safety and effectiveness for the referenced brand-name drug, and thus, in theory, the sponsor of the generic does not have to provide its own clinical studies to demonstrate the generic drug product's safety and effectiveness.

FDA recognizes the public policy benefit of generic competition but has understandably embraced safeguards to protect the public from generics that do not meet quality standards and/or are not objectively bioequivalent. Bioequivalence of typical drugs can be evaluated in a straightforward manner by use of standard analytical methods as well as comparison of absorption and pharmacokinetic parameters. However, complex products such a those utilizing liposomal encapsulation offer unique challenges in establishing bioequivalence since plasma concentrations of the active ingredient are not necessarily related to its availability at the target tissue due to the unique drug-release characteristics of the liposomal itself. FDA's mandate that generics must be demonstrably bioequivalent has been readily met by oral dosage forms, as well as intravenous dosage forms of many small molecule drugs, but presents unprecedented issues for prospective manufacturers of generic pegylated liposomal doxorubicin due to the unique pharmacokinetics (PK) and distribution provided by the liposome.

Previous studies have shown that plasma PK alone does not demonstrate bioequivalence for PLD. Clinical PK measurements of PLD indicate that the vast majority of the liposome's active ingredient, doxorubicin, remains encapsulated while the liposomes are in the blood stream and thus would not be bioavailable. The concentrations of both encapsulated and unencapsulated doxorubicin were measured in plasma of solid tumor patients for 30-day period following a single dose of PLD. Although the rates of clearance are similar for encapsulated and unencapsulated drug, the curve representing the plasma amount of encapsulated drug is three-orders of magnitude higher than that of the unencapsulated curve, indicating the vast majority of the drug remains encapsulated in the liposomes and is not released in the blood compartment. The lag time for the appearance of the major metabolite of doxorubicin, doxorubicinol, in plasma of patients who received PLD indicates that the free drug released from the liposomes was not available in plasma. The lack of doxorubicinol in plasma during the first few days after injection, the period during which total doxorubicin levels in plasma are highest, suggests that the drug is not available in free form (Hilger, Richly et al. 2001 and 2005). Thus, the plasma levels of PLD cannot be expected to serve as the sole surrogate of tissue levels at the drug's site of action as is the case with oral dosage forms.

Tumor uptake of doxorubicin after PLD administration has been contemplated as a surrogate parameter for demonstrating bioavailability in tissue. However, validity of this approach is lacking. Lack of clinical evidence of tumor uptake of PLD is related to the difficulty of measuring drug concentration in tissue. Patients treated with PLD typically have advance disease and bipsy in this setting can be invasive. Ad hoc radio-labeling methods have been reported for PLD (Koukourakis, et al. 2000), but these may change the product's performance, have not been validated and do not inform on the bioavailability of doxorubicin itself. Drug may be dispersed heterogeneously in tumor tissue. Kinetics and dose response relationships would be the most informative, but capturing such data would require multiple biopsies over a several day period. Reaching statistical power for a comparison of two similar products would be difficult. Different tumors, even of the same type, appear to accumulate PLD to different levels so the clinical significance of tumor uptake data is unknown (Harrington, Mohammadtaghi et al. 2001). Moreover, measurement of total drug does not inform on the bioavailable fraction, and separation of the encapsulated vs. released fraction in biopsy tissue is technically challenging. The invasiveness, technical difficulty, lack of sensitivity and precision and potential heterogeneity of response would argue against tumor uptake as a useful surrogate of PLD bioavailability.

According to the FDA 2007, because liposome products target specific tissues, the plasma concentration may not be related to the concentration of drugs at these specific tissues, and work is needed on novel bioequivalence methods to determine if two liposomes with the same composition but produced by different manufacturers have the same therapeutic profile (e.g., by assessing how they deliver drug to relevant tissues).

Previously reported clinical pharmacokinetic evidence suggests that following PLD administration to rodents, dogs and human patients, the vast majority of doxorubicin remains liposome-encapsulated during residence time of the drug in plasma (Hilger 2005). Based on this understanding, the US FDA recognized that traditional pharmacokinetic approaches to demonstrating bioequivalence do not apply to such liposomes because plasma levels do not necessarily reflect the bioavailability of the drug at its intended sites of action. “. . . it appears that the conventional pharmacokinetic approach may not be suitable for assessment of bioavailability or bioequivalence [for currently available liposome products]” (Chen 2005).

The FDA has publicly called for innovation: “Because liposome products target specific tissues, the plasma concentration may not be related to the concentration of drugs at these specific tissues, and work is needed on novel bioequivalence methods to determine if two liposomes with the same composition but produced by different manufacturers have the same therapeutic profile (e.g., by assessing how they deliver drug to relevant tissues)” (FDA 2007).

Reported preclinical and clinical studies indicate that skin toxicity is associated with multiple courses of PLD in mice, dogs and human patients. Charrois and Allen explored the relationship between PLD dose-rate and skin toxicity in mice. Four weekly doses of PLD at 9 mg/kg resulted in accumulation of doxorubicin in cutaneous tissues of mice and development of PPE-like lesions; accumulation of doxorubicin in skin and paws of mice is a gradual process reaching a peak three days post injection (Charrois and Allen 2003).

The relationship between PLD exposure and PPE was also examined in dogs (Amantea, Newman et al. 1999). Skin lesion development is dose-dependent, cumulative and reversible. Doxorubicin is undetectable in plasma at the time of onset of skin toxicity suggesting the effect originates in the tissue compartment (i.e., skin). The model predicts that the highest concentration of drug in the skin caused the most severe lesions suggesting a cause-and-effect relationship; drug that becomes available (i.e., was released from the liposomes) in the extravascular compartment causes local toxicity. The dog model has been validated by clinical experience; it correctly predicts that a dose intensity of 10-12.5 mg/m² of PLD will be well tolerated in human patients.

Based on these prior-art preclinical and clinical findings, it is reasonable for one skilled in the art (including experts at FDA who contributed to the Agency's position on liposome bioequivalence) to conclude that following PLD administration, doxorubicin enters the skin, becomes locally bioavailable and, with repeated exposure, induces skin toxicity. However, it was not recognized that Doxil's unique pattern of skin toxicity could be used as means of quantifying doxorubicin bioavailability in tissue (namely skin) in the context of demonstrating bioequivalence between Doxil and a generic product.

The published reports provide no suggestion or motivation to use measurement of the incidence and severity of PPE as an in vivo bioassay of tissue bioavailability in place of or in addition to PK to meet the statutory requirements for bioequivalence set by FDA.

Although equivalence of PK would be established using existing methods, and the incidence/severity of PPE would be scored separately/independently using known toxicity criteria, there is no recognition in the published reports that combining the two would provide a showing of bioequivalence. Indeed, in the published reports, PPE is regarded as a dose-limiting toxicity that is followed during PLD therapy (or in the context of clinical trials) as a safety end point, used to adjust dosing if necessary. There is no motivation to combine evidence of non-bioavailability in plasma (PK) with PPE (as an indirect measure of bioavailability in skin) to demonstrate statutory bioequivalence.

### SUMMARY OF THE INVENTION

In one aspect, the present invention provides a method of determining bioequivalence between a reference brand-name drug and a generic drug product for administering to a subject, the method comprising: (a) determining pharmacokinetic parameters of the reference brand-name drug and the generic drug product; (b) determining toxicity associated with administration of the reference brand-name drug and the generic drug product; (c) comparing the pharmacokinetic parameters and the toxicity profiles of the reference brand-name drug and the generic drug product; wherein equivalent pharmacokinetic parameters combined with equivalent toxicity profiles of the reference brand-name drug and the generic drug product indicate bioequivalence of the reference brand-name drug and the generic drug product when administered to the subject.

In some embodiments, the generic drug is a pegylated liposomal doxorubicin (PLD). In some embodiments, the reference brand-name drug is Doxorubicin HCl Liposome Injection (Doxil). In some embodiments, the toxicity is skin toxicity. In some embodiments, the toxicity is palmar plantar erythrodysesthesia (PPE). The toxicity profile can be PPE severity grade and/or dose reductions or modifications caused by PPE. In some embodiments, PPE serves as an in-vivo bioassay indicative of tissue bioavailability of the generic drug product and the reference brand-name drug. In some embodiments, the pharmacokinetic parameter is selected from the group consisting of absorption rate constant, bioavailability, apparent volume of distribution, steady-state volume of distribution, unbound fraction, rate of elimination, clearance, renal clearance, metabolic clearance, fraction excreted unchanged, elimination rate constant, biologic half-life, maximum concentration, and area under the concentration vs. time curve (AUC). In some embodiments, levels of total doxorubicin and its metabolite, doxorubicinol in plasma and urine are measured.

In some embodiments, levels of liposome-associated and free doxorubicin in plasma are measured. In some embodiments, the proportion of liposome associated to free doxorubicin in plasma is an indication of bioavailability of doxorubicin in plasma. In some embodiments, a high proportion of liposome-associated doxorubicin is an indication of lack of bioavailability in plasma.

In some embodiments, the pharmacokinetic parameter is a delay in the appearance of doxorubicinol in plasma. In some embodiments, equivalent delay in the appearance of doxorubicinol in plasma of the drug product relative to the reference brand-name drug combined with equivalent AUC of doxorubicinol in plasma of the drug product relative to the reference brand-name drug indicate equivalent tissue bioavailability of the generic drug product relative to the reference brand-name drug. In some embodiments, equivalent PK parameters and tissue bioavailability of the drug product relative to the reference brand-name drug indicate bioequivalence between the drug product and the reference brand-name drug. In some embodiments, the bioequivalence is met if the 90% confidence interval (CI) of the mean Cmax, AUC₀₋ₜ₎ and AUC_{(0-∞)} of the generic drug product relative to the reference brand-name drug is within 80% to 125%. In some embodiments, the bioequivalence determined meets the requirements set by the FDA. In some embodiments, the subject is a mammal, for example, a human. In some embodiments, the method is used to identify a generic drug product having an effective bioequivalence to the reference brand-name drug for administering to a subject. In some embodiments, the method is used to identify generic PLD products having an effective bioequivalence to doxorubicin hydrochloric acid liposome injection (Doxil) for administering to a subject.

In another aspect, the present invention provides a method of assessing bioequivalence between a generic pegylated liposomal doxorubicin (PLD) product and a reference doxorubicin hydrochloric acid liposome injection (Doxil) for administering to a subject, the method comprising: (a) determining pharmacokinetic parameters of doxorubicin hydrochloric acid liposome injection (Doxil) and the generic PLD product; (b) determining toxicity associated with administration of doxorubicin hydrochloric acid liposome injection (Doxil) and the generic PLD product; and (c) comparing the pharmacokinetic parameters and the toxicity profiles of doxorubicin hydrochloric acid liposome injection (Doxil) and the generic PLD product; wherein equivalent pharmacokinetic parameters plus equivalent toxicity profiles of Doxil and the generic PLD product indicate bioequivalence of Doxil and the generic PLD product when administered to the subject.

In some embodiments, the toxicity is skin toxicity. In some embodiments, the toxicity is palmar plantar erythrodysesthesia (PPE). In some embodiments, the toxicity profile is PPE toxicity grade or dose reductions or delays caused by PPE. In some embodiments, PPE is indicative of tissue bioavailability of the generic PLD product and Doxil. In some embodiments, the pharmacokinetic parameter is selected from the group consisting of absorption rate constant, bioavailability, apparent volume of distribution, steady-state volume of distribution, unbound fraction, rate of elimination, clearance, renal clearance, metabolic clearance, fraction excreted unchanged, elimination rate constant, biologic half-life, maximum concentration, and Area under Curve. In some embodiments, levels of doxorubicin and its metabolite, doxorubicinol in plasma and urine are measured. In some embodiments, the pharmacokinetic parameter is a delay in the appearance of doxorubicinol in plasma. In some embodiments, the bioequivalence is met if the 90% confidence interval (Cl) of the mean Cmax, AUC₍₀₋ₜ₎ and AUC_{(0-∞)} of the generic drug product relative to the reference brand-name drug is within 80% to 125%. In some embodiments, the bioequivalence determined meets the requirements set by the FDA. In some embodiments, the subject is a mammal. In some embodiments, the subject is a human. In some embodiments, the method is used to identify generic PLD products having an effective bioequivalence to doxorubicin hydrochloric acid liposome injection (Doxil) for administering to a subject.

In another aspect, the present invention provides a kit for determining bioequivalence between doxorubicin hydrochloric acid liposome injection (Doxil) and a generic PLD product, the kit comprising: (a) a generic PLD product; (b) reagents for performing in vitro and in vivo pharmacokinetic assays; and (c) instructions for using the kit. In some embodiments, the kit further comprises more than one generic PLD product. In some embodiments, the kit further comprises an instrument for performing in vivo pharmacokinetic study. In some embodiments, the kit further comprises a computer readable program or software for performing pharmacokinetic and/or statistical analysis.

In yet another aspect, the present invention provides a method of determining bioequivalence between an FDA-approved reference drug product and a pharmaceutically equivalent product that is produced using a manufacturing change relative to the reference product, the method comprising: (a) determining pharmacokinetic parameters of the reference drug and the pharmaceutically equivalent product; (b) determining toxicity associated with administration of the reference drug and the pharmaceutically equivalent product; and (c) comparing the pharmacokinetic parameters and the toxicity profiles of the reference drug and the pharmaceutically equivalent product; wherein equivalent pharmacokinetic parameters combined with equivalent toxicity profiles of the reference drug and a pharmaceutically equivalent product indicate bioequivalence of the reference drug with the pharmaceutically equivalent product made with a manufacturing change.

In some embodiments, the manufacturing change is a new manufacturing site and/or equipment. In some embodiments, the manufacturing change is an alternative process or process step. In some embodiments, the manufacturing change is a new source of raw materials. In some embodiments, the pharmaceutically equivalent drug is a pegylated liposomal doxorubicin (PLD). In some embodiments, the reference drug is Doxorubicin HCl Liposome Injection (Doxil). In some embodiments, the toxicity is skin toxicity. In some embodiments, the toxicity is palmar plantar erythrodysesthesia (PPE). In some embodiments, the toxicity profile is PPE toxicity grade and/or dose modifications or reductions caused by PPE. In some embodiments, PPE is indicative of tissue bioavailability of the pharmaceutically equivalent product and the reference drug. In some embodiments, the pharmacokinetic parameter is selected from the group consisting of absorption rate constant, bioavailability, apparent volume of distribution, steady-state volume of distribution, unbound fraction, rate of elimination, clearance, renal clearance, metabolic clearance, fraction excreted unchanged, elimination rate constant, biologic half-life, maximum concentration, and area under the concentration vs. time curve (AUC). In some embodiments, levels of doxorubicin and its metabolite, doxorubicinol in plasma and urine are measured. In some embodiments, the pharmacokinetic parameter is a delay in the appearance of doxorubicinol in plasma. In some embodiments, the bioequivalence is met if 90% confidence interval (CI) of the mean Cmax, AUC₍₀₋ₜ₎ and AUC_{(0-∞)} of the pharmaceutically equivalent drug product relative to the reference drug is within 80% to 125%. In some embodiments, the bioequivalence determined meets the requirements set by the FDA.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The present invention provides methods for determining bioequivalence between a generic drug product and a reference commerical product. In some embodiments, the present invention provides a method for determining bioequivalence between a reference doxorubicin hydrochloric acid (HCl) liposome injection product, Doxil, and a generic pegylated liposomal doxorubicin product, wherein the bioavailability and tissue distribution information determined using the subject methods satisfy the statutory requirements provided by the U.S. Food and Drug Administration (FDA). In some embodiments, the method of the present invention includes measuring the pharmacokinetic parameters plus toxicity associated with administration of a drug, for example, doxorubicin. In some embodiments, the toxicity associated with a drug such as doxorubicin is skin toxicity, such as palmar plantar erythrodysesthesi (PPE). In some embodiments, PPE can be used as a novel surrogate marker for determining tissue bioavailability of a drug such as doxorubicin. In some embodiments, an equivalent induction of toxicity such as PPE associated with administration of drugs is indicative of equivalent tissue bioavailability of the drug. The method of the present invention can be used to identify generic drugs that show bioequivalence to a reference commercial drug with parameters sufficient to meet the statutory requirements set by the FDA. The methods of the present invention reduce the analysis time and the costs otherwise associated with the evaluation of bioequivalence between a commercial brand-name drug and a generic drug, for example, between Doxil and a generic pegylated liposomal doxorubicin product.

Bioequivalence is a term in pharmacokinetics used to assess the expected in vivo biological equivalence of two proprietary preparations of a drug. If two products are said to be bioequivalent it means that they would be expected to be, for all intents and purposes, the same. Birkett (2003) defined bioequivalence by stating that, “two pharmaceutical products are bioequivalent if they are pharmaceutically equivalent and their bioavailabilities (rate and extent of availability) after administration in the same molar dose are similar to such a degree that their effects, with respect to both efficacy and safety, can be expected to be essentially the same. Pharmaceutical equivalence implies the same amount of the same active substance(s), in the same dosage form, for the same route of administration and meeting the same or comparable standards.” The term “bioequivalence” (BE) as used herein refers to the definition provided by the United States Food and Drug Administration (FDA), which recites, “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study” (FDA, 2003). According to the guideline provided by the U.S. FDA to the industry for the determination of bioequivalence (BE) for oral dosage forms, “in BE studies, an applicant compares the systemic exposure profile of a test drug product to that of a reference drug product. For two orally administered drug products to be bioequivalent, the active drug ingredient or active moiety in the test product should exhibit the same rate and extent of absorption as the reference drug product.” In some embodiments, the bioequivalence is defined as having been achieved if the 90% confidence interval (CI) of the mean maximum drug concentration (Cmax), and the area under the time-concentration curve (AUC) of the generic formulation of a drug, for example, a generic pegylated liposomal doxorubicin, relative to a reference brand-name formulation, for example, Doxil (doxorubicin hydrochloric acid liposome injection) is within about 80%, 85%, 90%, 95%, 100%, 105%, 110%, 115%, 120%, or 125%.

In general, to determine bioequivalence, for example, between two products such as a commercially-available Brand-name product and a potential to-be-marketed Generic product, pharmacokinetic studies are conducted whereby each of the preparations are administered in a cross-over study to volunteer subjects, generally healthy individuals but occasionally in patients. Serum/plasma samples are obtained at regular intervals and assayed for parent drug (or occasionally metabolite) concentration. Occasionally, blood concentration levels are neither feasible nor possible to compare the two products (e.g. inhaled corticosteroids), then pharmacodynamic endpoints rather than pharmacokinetic endpoints are used for comparison. For a pharmacokinetic comparison, the plasma concentration data are used to assess key pharmacokinetic parameters such as area under the curve (AUC), peak concentration (Cₘₐₓ), time to peak concentration (Tₘₐₓ), and absorption lag time (t_{lag}). Testing should be conducted at several different doses, especially when the drug displays non-linear pharmacokinetics. In addition to data from bioequivalence studies, other data may need to be submitted to meet regulatory equirements for bioequivalence. Such evidence may include analytical method validation and in vitro-in vivo correlation studies.

The term “bioavailability” as used herein refers to the rate and extent to which the active ingredient or active moiety is absorbed from a drug product and becomes available at the site of action. For drug products that are not intended to be absorbed into the bloodstream, bioavailability may be assessed by measurements intended to reflect the rate and extent to which the active ingredient or active moiety becomes available at the site of action. As part of Investigational New Drug (IND) and New Drug Applications (NDA) for orally administered drug products, BA studies focus on determining the process by which a drug is released from the oral dosage form and moves to the site of action. BA data provide an estimate of the fraction of the drug absorbed, as well as its subsequent distribution and elimination. BA can be generally documented by a systemic exposure profile obtained by measuring drug and/or metabolite concentration in the systemic circulation over time. The systemic exposure profile determined during clinical trials in the IND period can serve as a benchmark for subsequent BE studies.

In some embodiments, the methods of the present invention measure pharmacokinetic parameters for determining bioequivalence. Pharmacokinetics, abbreviated as “PK”, is to determinine the fate of substances administered externally to a living organism. In practice, PK study is applied mainly to drug substances, though in principle it concerns itself with all manner of compounds ingested or otherwise delivered externally to an organism, such as nutrients, metabolites, hormones, toxins, etc. Pharmacokinetics is often studied in conjunction with pharmacodynamics. Pharmacodynamics explores what a drug does to the body, whereas pharmacokinetics explores what the body does to the drug. Pharmacokinetics includes the study of the mechanisms of absorption and distribution of an administered drug, the rate at which a drug action begins and the duration of the effect, the chemical changes of the substance in the body (e.g. metabolism by enzymes) and the effects and routes of excretion of the metabolites of the drug. Pharmacokinetics is divided into several areas which include the extent and rate of absorption, distribution, metabolism and excretion. Pharmacokinetics describes how the body affects a specific drug after administration. Pharmacokinetic properties of drugs may be affected by elements such as the site of administration, the concentration in which the drug is administered and the dosage form (immediate or controlled release for example). These may affect the absorption rate. An important pharmacokinetic parameter is the biological half-life or elimination half life of a substance, which is the time it takes for a substance (drug, radioactive nuclide, or other) to lose half of its pharmacologic, physiologic, or radiologic activity.

Pharmacokinetic analysis is typically performed by noncompartmental (model independent) or compartmental methods. Noncompartmental methods estimate the exposure to a drug by estimating the area under the curve of a concentration-time graph. Compartmental methods estimate the concentration-time graph using kinetic models. Compartment-free methods do not assume any specific compartmental model and produce accurate results also acceptable for bioequivalence studies.

Noncompartmental PK analysis is highly dependent on estimation of total drug exposure. Total drug exposure is most often estimated by Area Under the Curve (AUC) methods, with the trapezoidal rule (numerical differential equations) the most common area estimation method. Due to the dependence of the length of ‘x’ in the trapezoidal rule, the area estimation is highly dependent on the blood/plasma sampling schedule. That is, the closer the time points are, the closer the trapezoids are to the actual shape of the concentration-time curve. Compartmental PK analysis uses kinetic models to describe and predict the concentration-time curve. The advantage of compartmental to some noncompartmental analysis is the ability to predict the concentration at any time. The disadvantage is the difficulty in developing and validating the proper model. Compartment-free modeling based on curve stripping does not suffer this limitation. “PK Solutions” is an easy-to-use, industry standard software that produces both noncompartmental as well as compartment-free results suitable for research and education. The simplest PK compartmental model is the one-compartmental PK model with IV bolus administration and first-order elimination. The more complex PK models rely on the use of physiological information to ease development and validation. Bioanalytical methods are necessary to construct a concentration-time profile. Chemical techniques are employed to measure the concentration of drugs in biological matrix, most often plasma. Proper bioanalytical methods used in PK studies are known to one skilled in the art (See Pharmacokinetics (2006) In Mosby's Dictionary of Medicine, Nursing, & Health Professions. Philadelphia, Pa.: Elsevier Health Sciences).

In some embodiments, the present invention evaluates pharmacokinetic parameters including but not limited to the relative area under the concentration versus time curve (AUC) for a drug, for example, doxorubicin and/or doxorubicinol. In one example, the PK parameter includes a time course in the appearance of a drug, for example, doxorubicinol in plasma. A further embodiment measures the proportion of doxorubicin in plasma that is liposome-encapsulated vs. total doxorubicin and/or free (unencapsulated). The finding that the vast majority of doxorubicin is liposome-encapsulated during the residence time of total doxrubicin in plama indicates the lack of bioavailability in plasma; the drug remains sequested in the liposome while residing in the blood stream. A delay in the appearance of doxorubicinol in plasma is indicative of doxorubicin's bioavailability in tissues. In some embodiments, an equivalent delay in the appearance of doxorubicinol in plasma of the drug product relative to the reference brand-name drug, plus equivalent AUC of doxorubicinol in plasma can be combined to demonstrate bioequivalence between a generic pegylated liposomal doxorubicin (PLD) product and the doxorubicin hydrochloric acid (HCl) liposome injection product-Doxil. The finding that the vast amount of total plasma doxorubicin (as measured by PK analysis of for example plasma AUC of total doxorubicin) is liposome-encapsulated indicates lack of bioavaialbility of the drug in plasma. The delay in the appearance of doxorubicinol confirms lack of bioavailabiliaty of free doxorubicin in plasma during the first few days after injection; the metabolite would appear immediately if doxorubicin were to become bioavailable in plasma because free doxorubicin in plasma immediately distributes to tissues, enters cells, is metabolized to doxorubicinol and is eliminated via the bloodstream. The delayed appearance of doxorubicinol indictaes that the liposome-encapsulated doxorubicin eventually becomes avaialable in tissues presumably as doxorubicin is released from the liposomes that have entered tissues, metabolized intracellularly and exits the tissue entering the central (blood) compartment. Thus, when the drug product and the reference brand-name drug are compared, the finding of equivalent total (and/or liposome-encapsulated) doxorubicin AUC, plus equivalent delay in the appaerance of doxorubicinol plus equivalent doxorubicinol AUC indcates equivalent tissue bioavialbility and thus bioequivalence.

In the case of oral dosage forms, PK measurements of the active ingredient (and/or its metabolites) in an accessible biological matrix (such as whole blood or plasma) serves as a surrogate for the demonstration of BE; the rate at which the active drug enters and leaves the bloodstream reflects its availability at the intended site of action, even though the actual concentration at the site is not measured. To measure product quality BA and establish BE, reliance on pharmacokinetic measurements may be viewed as a bioassay that assesses release of the drug substance from the drug product into the systemic circulation. For example, the blood levels of central nervous system (CNS) drug, such as diazepam, is recognized to reflect the drug's bioavailability in the brain.

In some embodiments, the methods of the present invention measure toxicity associated with administration of a drug for determining bioequivalence. Toxicokinetics as used herein refers to the application of pharmacokinetics to determine the relationship between the systemic exposure of a compound in experimental animals and its toxicity. In some embodiments, toxicity parameters including but not limited to the incidence and serevity of toxicity associated with administration of a drug are measured for determining the bioavailability and tissue distribution of the drug. In some embodiments, the bioavailabilities of the same drug can be compared to evaluate various formulations and/or routes of administration, thereby determining the bioequivalence between drugs of different formulations and/or routes of administration, or between a generic drug product and an established brand-name or patented drug product. The toxicities associated with a drug can include but are not limited to gastrointestinal toxicity, neurotoxicity, skin toxicity, hepatotoxicity, cardiotoxicity, myotoxicity, pulmonary toxicity, blood toxicity, visual toxicity, ototoxicity, nephrotoxicity, urinary tract toxicity, and toxicity associated with the reproductive system. In a specific example, the present invention provides a method of determining bioequivalence between a generic pegylated liposomal doxorubicin (PLD) product and a reference brand-name product, i.e. doxorubicin hydrochloric acid (HCl) liposome injection product Doxil, which includes a comparison of the relative incidence and severity of skin toxicity. Examples of skin toxicity that can be used as a parameter for determining bioequivalence of the present invention include but are not limited to palmer plantar erythrodysesthesia (PPE), mucositis and stomatitis.

A number of cytotoxic drugs have been reported to cause PPE including but not limited to PLD, 5-fluorouracil, capecitabine, and cytarabine. The initial symptoms of PPE are dysesthesia and tingling in the palms, fingers and soles of feet and erythema, which may progress to burning pain with dryness, cracking, desquamation, ulceration and edema. Palms of the hands are more frequently affected than soles of the feet. Histologically, vacuolar necrosis of basal keratinocytes and the lower third of the epidermis are seen in lesion biopsies. This condition is painful and distressing to patients and in some cases results in patients not being able to work or perform normal daily activities. PPE can also result in treatment interruptions that may impact the efficacy of the treatment regimen (Webster-Gandy, How et al. 2007; von Moos, Thuerlimann et al, 2008). The grading scale and management scheme for PPE extracted from PLD labeling is presented in Table 1. Grade 3 and 4 PPE are clearly distinguishable from Grade 1-2, the former having serious adverse impact on patients' daily lives. Dose delays and reductions, which may help to alleviate PPE, can compromise therapy.

TABLE 1
PPE toxicity grade and intervention algorithm

| Toxicity grade | | Dose adjustment |
|---|---|---|
| 1. | Mild erythema, swelling, or desquamation | Redose unless patient has experienced previous |
| | not interfering with daily activities | grade 3-4 toxicity. If so, delay up to 2 weeks and |
| | | decrease dose by 25%. Return to original dose |
| | | interval. |
| 2. | Erythema, desquamation, or swelling | Delay dose up to 2 weeks or until resolved to grade |
| | interfering with, but not precluding normal | 0-1. If after 2 weeks there is no resolution PLD |
| | physical activities; small blisters or | should be discontinued. |
| | ulcerations <2 cm in diameter | |
| 3. | Blistering, ulceration, or swelling | Delay dose up to 2 weeks or until resolved to grade |
| | interfering with walking or normal daily | 0-1. Decrease dose by 25% and return to original |
| | activities; cannot wear regular clothing | dose interval. If after 2 weeks there is no resolution |
| | | PLD should be discontinued. |
| 4. | Diffuse or local process causing infectious | Delay dose up to 2 weeks or until resolved to grade |
| | complications or a bed ridden state of | 0-1. Decrease dose by 25% and return to original |
| | hospitalization | dose interval. If after 2 weeks there is no resolution |
| | | PLD should be discontinued. |

In some embodiments, PPE is an indicator of skin toxicity associated with a drug, for example, doxorubicin, for determining bioequivalence of a generic PLD product and the reference product Doxil. In human patients PPE has rarely been reported as a major toxic effect of doxorubicin itself, although it has been reported in patients receiving dose-dense doxorubicin therapy (120 mg/m² Q3 weeks) (Ellis, Livingston et al. 2002) and among a series of advanced sarcoma patents who received continuous infusion of doxorubicin for a mean of 1I18 days and cumulative doses of up to one gram (Samuels, Vogeizang et al. 1987). Results of a randomized trial comparing the safety and efficacy of Doxil and doxorubicin confirm that, in the dose-range and infusion regimen typically used clinically, PPE is rarely seen following single-agent doxorubicin (O'Brien, Wigler et al. 2004). Thus, IV infusion of doxorubicin alone (for up to 60 min) at a dose of up to 60 mg/m² every 3 weeks (dose rate=20 mg/m²/week) does not typically cause clinically significant PPE.

In dogs the occurrence of PPE is associated more frequently with pegylated liposomal doxorubicin therapy than with nonliposomal doxorubicin therapy, at least when the latter is administered as a short-term infusion, and appears to be related to dose schedule. Treatment with 1.0 mg/kg pegylated liposomal doxorubicin once every 3 weeks produced only minor symptoms of dermal irritation, whereas weekly treatments with the same dose produced lesions so severe that treatment had to be interrupted, demonstrating the close relationship of PPE to dose interval. In general, pegylated liposomal doxorubicin-related PPE occur when plasma concentrations are no longer detectable, suggesting that the effect originates in an extravascular compartment (Amantea et al. 1999).

Previous studies have demonstrated that the profile of toxic effects of Doxil to the skin reflects its unique pharmacokinetics and tissue distribution. These skin reactions vary significantly from those associated with doxorubicin in non-liposome-encapsulated form (Lotem, Hubert et al. 2000). Doxorubicin encapsulated in non-pegylated liposomes, which do not circulate for a long period of time because they are taken up by the mononuclear phagocyte system (MPS) e.g. macrophages residing in the tissues such as liver and spleen, within a few minutes of injection, given at a dose of 75 mg/m²/every 3 weeks (dose rate=25 mg/m²/week), does not cause significant PPE (1 grade 2 PPE was reported out of 108 patients) (Harris, Batist et al. 2002), suggesting that PPE is a consequence of the unique tissue distribution of pegylated liposomal form of doxorubicin (PLD). In some embodiments of the present invention, equivalent PPE in the reference product Doxil and the generic PLD product can be used for demonstrating equivalent tissue bioavailability.

The methods of the present invention can be applied to a subject. In some embodiments, the subject is a mammal including but not limited to mouse, rat, rabbit, dog, cat, monkey, and the like. In a preferred embodiment, the subject is a human. In some embodiments, the etiology, onset and dose response relationships of PLD-induced skin toxicity in dogs is similar to those in humans and a validated model exists to predict the relationship between dose rate and PPE in humans.

The methods of the present invention find utility in demonstrating bioequivalence between a reference commercial brand-name product and a generic product using both pharmacokinetic and toxicity parameters. In one example, the method of the present invention can be used to demonstrate bioequivalence between doxorubicin hydrochloric acid liposome injection product Doxil and a generic pegylated liposomal doxorubicin product based on equivalent PK parameters and equivalent PPE manifestations. However, it should be noted that the method of the present invention encompasses assessing bioequivalence between a generic product and any corresponding commercial brand-name product, wherein equivalent pharmacokinetic parameters combined with an equivalent toxicity profile of the commerical brand-name drug and the generic product indicate bioequivalence of the commerical brand-name drug and the generic product when administered to a subject.

In another aspect, the present invention also includes a kit for performing the assays to determine the bioequivalence between a commerical brand-name drug and a generic product. In some embodiments, a subject kit contains liposomes and pegylated liposomal doxorubicin. In one embodiment, the kit further includes various salts, dextrose, fatty alcohols, fatty acids, and/or cholesterol esters of any other pharmaceutically acceptable excipients which may affect incorporation of the drug, e.g. doxorubicin in the liposomes. In some embodiments, the subject kit further comprises reagents for carrying out pharmacokinetic assays, for example, reagents for processing blood and measuring drug concentrations in plasma. In some embodiments, the subject kit further comprises instruments such as syringes, needles, and catheters for carrying out in vivo PK studies for determining bioequivalence.

A subject kit can further include, if desired, one or more of various conventional components, such as, for example, containers with one or more buffers, detection reagents, enzymes or antibodies. Printed instructions, either as inserts or as labels, indicating quantities of the components to be used and guidelines for their use, can also be included in the kit. In the present disclosure it should be understood that the specified materials and conditions are important in practicing the invention but that unspecified materials and conditions are not excluded so long as they do not prevent the benefits of the invention from being realized. Exemplary embodiments of the methods of the invention are described above in detail.

The subject kits may also contain reference samples containing more than one generic drug product or a generic drug product that has been tested to show bioequivalence with a brand-name product, where the dilution series of the tested generic product represents a range of appropriate standards with which a user of the kit can compare their results and estimate the PK parameters of a generic product being tested. Fluorescence, color, or autoradiological reagents that may aim in any of the in vitro or in vivo assays in determining bioequivalence may also be provided by the kit.

In addition to the above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Also provided by the subject invention are kits including at least a computer readable medium including programming and software. In some embodiments, the kit includes software for performing pharmacokinetic and statistical analyses. For example, the population pharmacokinetic values can be determined by IT2S, a program developed using modules from the program package, ADAPT II. The computer readable instructions may include installation or setup directions. The instructions may include directions for use of the-invention with options or combinations of options as described above. In certain embodiments, the instructions include both types of information.

Manufacturing changes may alter the performance of a complex drug delivery system and thus when such a change is introduced the sponsor must show the drug product produced is bioequivalent to the original reference product. Also provided by the subject invention is a method of determining bioequivalence between an FDA-approved reference drug product and a product that is pharmaceutically equivalent but produced using a manufacturing change. Manufacturing changes may include the inrtruction of alternative process steps or changes in existing process steps, new equipment, a new manufacturing site and alternate raw materials.

### EXAMPLES

### Example 1

### Dose Comparative Toxicity of Generic PLD and Doxil in Dogs

In the pre-clinical studies disclosed herein, the levels of doxorubicin and doxorubicinol, the primary metabolite of doxorubicin, are measured in plasma and urine. Bioequivlance as used in these studies refers to parameters having been achieved if the 90% confidence interval (Cl) of the mean Cmax, AUC₍₀₋ₜ₎ and AUC_{(0-∞)} of the generic formulation relative to reference brand-name formulation is within 80% to 125%. Assays to be utilized in these studies are fully validated and capable of detecting low levels of doxorubicin and doxorubicinol in plasma and tissues.

This example is to determine the relative tissue bioavailability of a generic PLD and the reference commercial proiduct, doxorubicin HCl liposome injection, known as DOXIL® (US) and CAELYX® (EU), based on the development of skin toxicity, i.e. PPE. A repeated dose comparative toxicity and pharmacokinetic study of the generic PLD product and Doxil is conducted in dogs. Dogs are treated with 1.0 mg/kg of either the generic PLD product or Doxil weekly for 4 consecutive weeks. The study design provides, blood sample collection times and PPE scoring scale are based on Amantea et al. (1999), which is herein incorporated by reference in its entirety. Specifically, twenty dogs are assigned to each of the generic PLD product or Doxil groups (2/sex/ group) that receive intravenous doses of 0.5 mg/kg q1, 2, or 4 weeks; 1.0 mg/kg q2weeks; or 1.5 mg/kg q4weeks. Each group is treated for a total of 12 weeks. Blood is collected for HPLC analysis of doxorubicin concentration pre-dose and periodically up to 120 h after dosing three times during treatment. Dermal lesions are scored twice weekly at six regions of each dog using a 0-6 severity scale; maximum total severity is thus 36. PPE score data are modeled using an approach in which the % probability of PPE is related to a hypothetical effect site by a series of Hill-type functions. Dog pharmacokinetics is best modeled as a one-compartment open model. Cₘₐₓ increases linearly with dose. Total plasma clearance (CLt) decreases with repeated doses.

Dogs treated with both the generic PLD product and Doxil develop symptoms reminiscent of dermal toxicity seen in patients. As in patients, skin lesion development is dose-dependent, cumulative and reversible. A delay (or hysteresis) is seen between the peak plasma concentration and the onset of lesion development. The drug is undetectable in plasma at the time of onset of skin toxicity suggesting the effect originates in the extravascular compartment. A two-compartment pharmacodynamic model, which correctly predicted 97% of the observed lesion severity, is adapted to establish the relationship of lesion severity to dose intensity (a measure of drug exposure incorporating the effect of both dose level and dosing frequency, which can be expressed in units of mg/kg/week). The model predicts the amount of drug entering a hypothetical effect compartment (e.g. skin) and demonstrates that maximal PPE is positively correlated with dose intensity, the major factor that affects the incidence and severity of dermal lesions. The highest concentration of drug predicted in the extravascular compartment causes the most severe lesions suggesting a cause-and-effect relationship; drug that becomes available in the extravascular compartment causes local toxicity.

Administrations of both the generic PLD and Doxil result in dose-dependent skin toxicity PPE, and the drug must exit the liposome and enter skin in order to express this toxicity. Thus, an equivalent induction of PPE shows that doxorubicin becomes bioavailable in the skin to the same extent and indicates equivalent bioavailability of the generic PLD product and Doxil in dogs. The model can be used to predict acceptable dose intensities in humans utilizing body surface area conversion factors and comparative AUCs for dogs and humans. The data derived from the experiments are also fit to the two-compartment pharmacodynamic model developed by Amantea et al. (1999). Briefly, the fitted pharmacokinetic model parameters include the volume of distribution at steady state (Vₛₛ) and total plasma clearance (CLt), which is modeled as a linear (first order) eliminative process. In addition, Vss and CLt are allowed to vary between each pharmacokinetic sampling period to determine if the pharmacokinetics in the dogs changes over the duration of the study. A good or equivalent fit for Doxil and the generic PLD product demonstrates bioequivalence. This example demonstrates that PPE can be used as a novel surrogate marker of tissue bioavailability.

### Example 2

### Comparative Studies of Generic PLD Product and Doxil in Tumor Tissues

In one aspect, this example compares the anti-tumor activity of Doxil and the generic PLD in the syngeneic CT26 murine colon tumor model in BALB/c mice and the human xenograft HEY ovarian carcinoma model in nude mice.

Liposomes can be physically trapped in the spaces between tumor cells (the interstitium) following entry into tumors. Retention of liposomal drugs in the tumor may be related to the observation that tumors generally lack lymphatic vessels, which in normal tissues serve to clear entering foreign particles. Liposomes entrapped in the tumor eventually release the drug contained inside that the free drug then enters and kills the nearby tumor cells. Thus, tumor cell uptake and the anti-tumor efficacy of Doxil and the generic PLD can be used as a parameter to determine tissue availability of the free drug, doxorubicin. The rationale is that comparable anti-tumor activity of Doxil and the generic PLD suggests equivalent bioavailability in tissues. Due to the inherent variability in the tumor models, more mice are used per dose group and parallel replicate studies are undertaken. An orthotopic ovarian model is also utilized for comparing the anti-tumor activity of Doxil and the generic PLD. These models provide preliminary comparative toxicity indicative of the drug tissue bioavailability, albeit in tumor-bearing animals.

In another aspect, this example compares drug release in tumor tissues using microdialysis. The rationale is to provide a direct measure of drug release rate from extravasated liposomes that in tumor tissue, which directly demonstrates equivalent release rates and, therefore, equivalent bioavailability. As described in Zamboni et al. (2004) for pegylated liposomal cisplatin, which is herein incorporated by reference, parameters such as clearance rate, area under the concentration-time curve, as well as half-life of the generic PLD product are compared those of Doxil. Coupled with the tumor tissue distribution studies disclosed herein, bioequivalence of Doxil and the generic PLD is demonstrated.

Single dose tissue distribution of Doxil and the generic PLD is also studied and compared in normal and tumor-bearing mice, as well as in normal dogs. The effect and disposition of Doxil and the generic PLD on selected tissues including bone marrow, duodenum, heart, kidney, liver, lungs, skin, spleen, and stomach are evaluated. An equivalent tissue distribution of Doxil and the generic PLD demonstrates equivalent bioavailability.

### Example 3

### Comparative Pharmacokinetics of Generic PLD and Doxil in Rats and Dogs

Comparative single dose pharmacokinetics of the generic PLD product and Doxil are measured in rats and dogs. Levels of doxorubicin and doxorubicinol are measured in encapsulated and unencapsulated doxorubicin. Urine and bile in rats are collected to evaluate the comparative excretion of the generic PLD product versus Doxil.

PK studies reported for PLD (Working and Dayan, 1996) and more recently by Charrois and Allen (2004) provide the foundation that similar plasma pharmacokinetics should be an acceptable and accurate surrogate for drug release rate, i.e., two formlations that have equivalent plasma pharmacokinetics also have similar drug release rates. This relationship is applicable for Doxil and the generic PLD product since the free unencapsulated doxorubicin is rapidly cleared from the plasma.

### Example 4

### Pharmacokinectic Study of Generic PLD and Doxil in Humans

After having conducted the preclinical studies described in Examples 1-3, a pilot human PK study with a safety lead-in dose for the generic PLD product is conducted. The first dose of the generic is given to 3 patients at half the recommended dose of the reference product, Doxil, (i.e., generic PLD given at 25 mg/m²) to limit the drug exposure and insure patient safety. The results show that the generic PLD product is well tolerated. Subsequently, two additional groups of 3 patients per group are given a single dose of either Doxil or the generic PLD product at 50 mg/m². Plasma samples are collected. The total, encapsulated vs. unencapsulated doxorubicin levels are measured using a published method known to one skilled in the art for separation of the released drug from the liposomes. The results show that both the generic PLD product and the reference drug Doxil are safe and retain >80% of the total doxorubicin at the 30 after injection, and that the clearance curves are similar thereafter suggesting that the majority of the drug in both products remains encapsulated during residence of the liposomes in plasma. The pilot PK study shows that equivalent amounts of the drug remain liposome-encapsulated in both Doxil and the generic PLD products at a selected time point, thereby demonstrating bioequivalence of the generic PLD product and Doxil in humans.

A definitive PK study is then carried out in humans to further evaluate the bioequivalence of the generic PLD product and Doxil. Since the pilot PK study shows that the majority of doxorubicin remains encapsulated and the free doxorubicin is not bioavailable in plasma (were it to be released, doxorubicin would rapidly leave the blood compartment and distribute to tissues), total doxorubicin level rather than the fraction of encapsulated vs. unencapsulated doxorubicin is measured in the definitive PK study. The definitive PK study is a crossover design to control for the inter-patient variation seen with liposomes. Adopting this approach, patients are assigned to either the reference Doxil group or the generic PLD group and receive a single 40-50 mg/m² dose. Following a 4-week washout period, each patient crosses over to the other product at the same dose. Total doxorubicin and doxorubicinol are quantified in plasma and urine over a several day period using validated analytical methodology. The results show similar levels of doxorubicinol in the plasma suggesting equivalent bioavailability, since the parent compound is not metabolized unless it has been released from the liposome. Similar or equivalent metabolite levels thus suggest equivalent bioavailability. A standard statistical treatment is applied. For example, equivalence can be defined as having been achieved if the 90% confidence interval (CI) of the mean Cmax, AUC₍₀₋ₜ₎ and AUC_{(0-∞)} of the generic formulation relative to the reference brand-name formulation (i e. Doxil) is within 80% to 125%. A comparison of PK parameters in patients receiving multiple doses of each product also may be considered since it is known that doxorubicin plasma clearance slows after multiple doses of PLD.

### Example 5

### Comparative Skin Toxicity of Generic PLD and Doxil in Humans

Skin toxicity studies comparing the generic PLD product and the reference product Doxil are conducted in human patients. Since the PK study shows that most of doxorubicin is encapsulated in blood and therefore not bioavailable in tissues, an indication of tissue bioavailability is needed to demonstrate bioequivalence in this case. The subject method of the present invention utilizes PPE as a surrogate indicator of tissue bioavailability. Thus in this example, PPE associated with administration of the generic PLD product and the reference product Doxil is compared in a randomized, blinded trail as a means to demonstrate the requisite bioavailability.

Solid tumor patients are randomly assigned to either the reference group or the generic PLD group. The number of patients per arm needed to show equivalence is based on the known incidence of PPE for the reference Doxil and the statistical power recommended by the U.S. FDA. The treatment is blinded, and each product is given at a dose of 40-50 mg/m² for 6 cycles. Comparison of the incidence and severity of PPE assessed regularly during the treatment is the primary end point of the study. Dose reductions or delays during a course of therapy are secondary endpoints. The standard statistical treatment is applied and equivalence is demonstrated when values fall within 90% confidence interval of the relative mean.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

Al-Batran, S. E., H. G. Meerpohl, et al. (2006). “Reduced incidence of severe pairiar-plantar erythrodysesthesia and mucositis in a prospective multicenter phase II trial with pegylated liposomal doxorubicin at 40 mg/m2 every 4 weeks in previously treated patients with metastatic breast cancer.” Oncology 70(2): 141-6.
Alberts, D. S., F. M. Muggia, et al. (2004). “Efficacy and safety of liposomal anthracyclines in phase I/II clinical trials.” Semin Oncol 31(6 Suppl 13): 53-90.
Amantea, M., M. S. Newman, et al. (1999). “Relationship of dose intensity to the induction of palmar-plantar erythrodysesthia by pegylated liposomal doxorubicin in dogs.” Hum Exp Toxicol 18(1): 17-26.
Campos, S. M., R. T. Penson, et al. (2001). “The clinical utility of liposomal doxorubicin in recurrent ovarian cancer.” Gynecol Oncol 81(2): 206-12.
Chen, C. (2005). Regulatory Considerations for Contolled Release Parenteral Drug Products: Liposomes ans Microscpheres. Injectable Disperse Systems: formulation, processing and performance. Burgess. Boca Raton, Taylor and Francis: 629-30.
Charrois, G. J. and T. M. Allen (2003). “Multiple injections of pegylated liposomal Doxorubicin: pharmacokinetics and therapeutic activity.” J Pharmacol Exp Ther 306(3): 1058-67.
Charrois, G. J. and T. M. Allen (2004). “Drug release rate influences the pharmacokinetics, biodistribution, therapeutic activity, and toxicity of pegylated liposomal doxorubicin formulations in murine breast cancer.” Biochim Biophys Acta 1663(1-2): 167-77.
Coleman, R. E., L. Biganzoli, et al. (2006). “A randomised phase 11 study of two different schedules of pegylated liposomal doxorubicin in metastatic breast cancer (EORTC-10993).” Eur J Cancer 42(7): 882-7.
Ellis, G. K., R. B. Livingston, et al. (2002). “Dose-dense anthracycline-based chemotherapy for node-positive breast cancer.” J Clin Oncol 20(17): 3637-43.
FDA (2003). Guidance for Industry Bioavailability and Bioequivalence Studies for Orally Administered Drug Products—General Considerations Center of Drug Evaluation and Reseach. [www.fda.gov/cder/guidance/2191 dft.pdf]
FDA (2007). “Critical Path Opportunities for Generic Drugs ” Office of Generic Drugs. [http://www.fda.gov/oc/initiatives/criticalpath/reports/generic.html#liposome]
Gordon, A. N., J. T. Fleagle, et al. (2001). “Recurrent epithelial ovarian carcinoma: a randomized phase III study of pegylated liposomal doxorubicin versus topotecan.” J Clin Oncol 19(14): 3312-22.
Harrington, K. J., S. Mohammadtaghi, et al. (2001). “Effective targeting of solid tumors in patients with locally advanced cancers by radiolabeled pegylated liposomes.” Clin Cancer Res 7(2): 243-54.
Harris, L., G. Batist, et al. (2002). “Liposome-encapsulated doxorubicin compared with conventional doxorubicin in a randomized multicenter trial as first-line therapy of metastatic breast carcinoma.” Cancer 94(1): 25-36.
Hilger, R. A., H. Richly, et al. (2005). “Pharmacokinetics (PK) of a liposomal encapsulated fraction containing doxorubicin and of doxorubicin released from the liposomal capsule after intravenous infusion of Caelyx/Doxil.” Int J Clin Pharmacol Ther 43(12): 588-9.
Hilger, R. A., H. Richly, et al. (2001). “Pharmacokinetics of Encapsulated as Well as Free Doxorubicin and its Metabolites After Intravenous Infusion of Caelyx.” Proc Am Soc Clin Oncol 20: 116a.
Johnson, J. a. (2008). “Ortho Biotech Files sNDA for DOXIL® for the Treatment of Advanced Breast Cancer.” [http://www.investor.jnj.com/mobile/releasedetail.cfm?ReleaseID=333558].
Keller, A. M.,,R. G. Mennel, et al. (2004). “Randomized phase III trial of pegylated liposomal doxorubicin versus vinorelbine or mitomycin C plus vinblastine in women with taxane-refractory advanced breast cancer.” J Clin Oncol 22(19): 3893-901.
Kim, R. J., G. Peterson, et al. (2005). “Skin toxicity associated with pegylated liposomal doxorubicin (40 mg/m2) in the treatment of gynecologic cancers.” Gynecol Oncol 97(2): 374-8.
Koukourakis, M. I., S. Koukouraki, et al. (2000). “High intratumoral accumulation of stealth liposomal doxorubicin in sarcomas--rationale for combination with radiotherapy.” Acta Oncol 39(2): 207-11.
Lorusso, D., A. Naldini, et al. (2004). “Phase II study of pegylated liposomal doxorubicin in heavily pretreated epithelial ovarian cancer patients. May a new treatment schedule improve toxicity profile?” Oncology 67(3-4): 243-9.
Lotem, M., A. Hubert, et al. (2000). “Skin toxic effects of polyethylene glycol-coated liposomal doxorubicin.” Arch Dermatol 136(12): 1475-80.
Maeda, H., J. Wu, et al. (2000). “Tumor vascular permeability and the EPR effect in macromolecular therapeutics: a review.” J Controlled Release 65(1-2): 271-84.
Martin, F. (2008). Pegylated Liposome Delvery of Chemotherapeutic Agents: Rationale and Clinical Benefit. N.Y., Informa Healthcare.
Martin, F. J. (1998). Clinical pharmacology and antitumor efficacy of DOXIL (pegylated liposomal doxorubicin). Medical Apnlications of Liposomes. D. D. Lasic, Papahadjopoulos, D. Amsterdam, Elsevier: 635-688.
O'Brien, M. E. (2008). “Single-agent treatment with pegylated liposomal doxorubicin for metastatic breast cancer.” Anticancer Drugs 19(1): 1-7.
O'Brien, M. E., N. Wigler, et al. (2004). “Reduced cardiotoxicity and comparable efficacy in a phase III trial of pegylated liposomal doxorubicin HCl (CAELYX/Doxil) versus conventional doxorubicin for first-line treatment of metastatic breast cancer.” Ann Oncol 15(3): 440-9.
Ranson, M. R., J. Carmichael, et al. (1997). “Treatment of advanced breast cancer with sterically stabilized liposomal doxorubicin: results of a multicenter phase II trial [see comments].” J Clin Oncol 15(10): 3185-91.
Samuels, B. L., N. J. Vogeizang, et al. (1987). “Continuous venous infusion of doxorubicin in advanced sarcomas.” Cancer Treat Rep 71(10): 971-2.
Schering-Plough (2008). “2Q Earnings Report.” [http://www.schering-plough.com/schering_plough/news/release.jsp?releaseID=1177275]
Sehouli, J., G. Oskay-Ozcelik, et al. (2006). “Biweekly pegylated liposomal doxorubicin in patients with relapsed ovarian cancer: results of a multicenter phase-II trial.” Ann Oncol 17(6): 957-61.
Tibotec Therapeutics/Division of Ortho Biotech Products.Bridgewater, N. (2005). “Doxil (Doxorubicin HCl Liposome Injection): Prescribing information”.
Uziely, B., S. Jeffers, et al. (1995). “Liposomal doxorubicin: antitumor activity and unique toxicities during two complementary phase I studies.” J Clin Oncol 13(7): 1777-85.
Vail, D. M., M. A. Amantea, et al. (2004). “Pegylated liposomal doxorubicin: proof of principle using preclinical animal models and pharmacokinetic studies.” Semin Oncol 31(6 Suppl 13): 16-35.
von Moos, R., B. J. Thuerlimann, et al. (2008). “Pegylated liposomal doxorubicin-associated hand-foot syndrome: recommendations of an international panel of experts.” Eur J Cancer 44(6): 781-90.
Webster-Gandy, J. D., C. How, et al. (2007). “Palmar-plantar erythrodysesthesia (PPE): a literature review with commentary on experience in a cancer centre.” Eur J Oncol Nurs 11(3): 238-46.

1. A method of determining bioequivalence between a reference brand-name drug and a generic drug product for administering to a subject, the method comprising:
a. determining pharmacokinetic parameters of the reference brand-name drug and the generic drug product
b. determining toxicity associated with administration of the reference brand-name drug and the generic drug product
c. comparing the pharmacokinetic parameters and the toxicity profiles of the reference brand-name drug and the generic drug product
wherein equivalent pharmacokinetic parameters combined with equivalent toxicity profiles of the reference brand-name drug and the generic drug product indicate bioequivalence of the reference brand-name drug and the generic drug product when administered to the subject.

2. The method of claim 1, wherein the generic drug is a pegylated liposomal doxorubicin (PLD).

3. The method of claim 1, wherein the reference brand-name drug is Doxorubicin HCl Liposome Injection (Doxil).

4. The method of claim 1, wherein the toxicity is skin toxicity.

5. The method of claim 1, wherein the toxicity is palmar plantar erythrodysesthesia (PPE).

6. The method of claim 1, wherein the toxicity profile is PPE severity grade, dose reduction or delays caused by PPE.

7. The method of claim 5 or 6, wherein PPE serves as an in-vivo bioassay indicative of tissue bioavailability of the generic drug product and the reference brand-name drug.

8. The method of claim 1, wherein the pharmacokinetic parameter is selected from the group consisting of absorption rate constant, bioavailability, apparent volume of distribution, steady-state volume of distribution, unbound fraction, rate of elimination, clearance, renal clearance, metabolic clearance, fraction excreted unchanged, elimination rate constant, biologic half-life, maximum concentration, and area under the concentration vs. time curve (AUC).

9. The method of claim 1, wherein levels of total doxorubicin and its metabolite, doxorubicinol in plasma and urine are measured.

10. A method of claim 9, wherein the levels of liposome-associated and free doxorubicin in plasma are measured.

11. A method of claim 10, wherein the proportion of liposome associated to free doxorubicin in plasma is an indication of bioavailability of doxorubicin in plasma.

12. A method of claim 11, wherein a high proportion of liposome-associated doxorubicin is an indication of lack of bioavailability in plasma.

13. The method of claim 1, wherein the pharmacokinetic parameter is a delay in the appearance of doxorubicinol in plasma.

14. A method of claim 13, wherein equivalent delay in the appearance of doxorubicinol in plasma of the drug product relative to the reference brand-name drug combined with equivalent AUC of doxorubicinol in plasma of the drug product relative to the reference brand-name drug indicate equivalent tissue bioavailability of the generic drug product relative to the reference brand-name drug.

15. A method of claims 8 and 14, wherein equivalent PK parameters and tissue bioavailability of the drug product relative to the reference brand-name drug indicate bioequivalence between the drug product and the reference brand-name drug.

16. The method of claim 1, wherein the bioequivalence is met if the 90% confidence interval (CI) of the mean Cmax, AUC(₀₋ₜ) and AUC(_{0-∞)} of the generic drug product relative to the reference brand-name drug is within 80% to 125%.

17. The method of claim 1, wherein the bioequivalence determined meets the requirements set by the FDA.

18. The method of claim 1, wherein the subject is a mammal.

19. The method of claim 1, wherein the subject is a human.

20. The method of claim 1, wherein the method is used to identify a generic drug product having an effective bioequivalence to the reference brand-name drug for administering to a subject.

21. The method of claim 1, wherein the method is used to identify generic PLD products having an effective bioequivalence to doxorubicin hydrochloric acid liposome injection (Doxil) for administering to a subject.

22. A method of assessing bioequivalence between a generic pegylated liposomal doxorubicin (PLD) product and a reference doxorubicin hydrochloric acid liposome injection (Doxil) for administering to a subject, the method comprising:
a. determining pharmacokinetic parameters of doxorubicin hydrochloric acid liposome injection (Doxil) and the generic PLD product
b. determining toxicity associated with administration of doxorubicin hydrochloric acid liposome injection (Doxil) and the generic PLD product
c. comparing the pharmacokinetic parameters and the toxicity profiles of doxorubicin hydrochloric acid liposome injection (Doxil) and the generic PLD product
wherein equivalent pharmacokinetic parameters plus equivalent toxicity profiles of Doxil and the generic PLD product indicate bioequivalence of Doxil and the generic PLD product when administered to the subject.

23. The method of claim 22, wherein the toxicity is skin toxicity.

24. The method of claim 22, wherein the toxicity is palmar plantar erythrodysesthesia (PPE).

25. The method of claim 22, wherein the toxicity profile is PPE toxicity grade or dose modifications.

26. The method of claim 24 or 25, wherein PPE is indicative of tissue bioavailability of the generic PLD product and Doxil.

27. The method of claim 22, wherein the pharmacokinetic parameter is selected from the group consisting of absorption rate constant, bioavailability, apparent volume of distribution, steady-state volume of distribution, unbound fraction, rate of elimination, clearance, renal clearance, metabolic clearance, fraction excreted unchanged, elimination rate constant, biologic half-life, maximum concentration, and Area under Curve.

28. The method of claim 22, wherein levels of doxorubicin and its metabolite, doxorubicinol in plasma and urine are measured.

29. The method of claim 22, wherein the pharmacokinetic parameter is a delay in the appearance of doxorubicinol in plasma.

30. The method of claim 22, wherein the bioequivalence is met if the 90% confidence interval (CI) of the mean Cmax, AUC(₀₋ₜ) and AUC(_{0-∞)} of the generic drug product relative to the reference brand-name drug is within 80% to 125%.

31. The method of claim 22, wherein the bioequivalence determined meets the requirements set by the FDA.

32. The method of claim 22, wherein the subject is a mammal.

33. The method of claim 22, wherein the subject is a human.

34. The method of claim 22, wherein the method is used to identify generic PLD products having an effective bioequivalence to doxorubicin hydrochloric acid liposome injection (Doxil) for administering to a subject.

35. A kit for determining bioequivalence between doxorubicin hydrochloric acid liposome injection (Doxil) and a generic PLD product, the kit comprising:
a. a generic PLD product
b. reagents for performing in vitro and in vivo pharmacokinetic assays
c. instructions for using the kit

36. The kit of claim 35 further comprising more than one generic PLD product.

37. The kit of claim 35 further comprising an instrument for performing in vivo pharmacokinetic study.

38. The kit of claim 35 further comprising a computer readable program or software for performing pharmacokinetic and/or statistical analysis.

39. A method of determining bioequivalence between an FDA-approved reference drug product and a pharmaceutically equivalent product that is produced using a manufacturing change relative to the reference product, the method comprising:
a. determining pharmacokinetic parameters of the reference drug and the pharmaceutically equivalent product
b. determining toxicity associated with administration of the reference drug and the pharmaceutically equivalent product
c. comparing the pharmacokinetic parameters and the toxicity profiles of the reference drug and the pharmaceutically equivalent product
wherein equivalent pharmacokinetic parameters combined with equivalent toxicity profiles of the reference drug and a pharmaceutically equivalent product indicate bioequivalence of the reference drug with the pharmaceutically equivalent product made with a manufacturing change.

40. The method of claim 39, wherein the manufacturing change is a new manufacturing site and/or equipment.

41. The method of claim 39, wherein the manufacturing change is an alternative process or process step.

42. The method of claim 39, wherein the manufacturing change is a new source of raw materials.

43. The method of claim 39, wherein the pharmaceutically equivalent drug is a pegylated liposomal doxorubicin (PLD).

44. The method of claim 39, wherein the reference drug is Doxorubicin HCl Liposome Injection (Doxil).

45. The method of claim 39, wherein the toxicity is skin toxicity.

46. The method of claim 39, wherein the toxicity is palmar plantar erythrodysesthesia (PPE).

47. The method of claim 39, wherein the toxicity profile is PPE toxicity grade or dose modifications.

48. The method of claim 46 or 47, wherein PPE is indicative of tissue bioavailability of the pharmaceutically equivalent product and the reference drug.

49. The method of claim 39, wherein the pharmacokinetic parameter is selected from the group consisting of absorption rate constant, bioavailability, apparent volume of distribution, steady-state volume of distribution, unbound fraction, rate of elimination, clearance, renal clearance, metabolic clearance, fraction excreted unchanged, elimination rate constant, biologic half-life, maximum concentration, and area under the concentration vs. time curve (AUC).

50. The method of claim 39, wherein levels of doxorubicin and its metabolite, doxorubicinol in plasma and urine are measured.

51. The method of claim 39, wherein the pharmacokinetic parameter is a delay in the appearance of doxorubicinol in plasma.

52. The method of claim 39, wherein the bioequivalence is met if 90% confidence interval (CI) of the mean Cmax, AUC(₀₋ₜ) and AUC(_{0-∞)} of the pharmaceutically equivalent drug product relative to the reference drug is within 80% to 125%.

53. The method of claim 39, wherein the bioequivalence determined meets the requirements set by the FDA.
